(19) United States
(12) Patent Application Publication
(10) Pub. No.: US 2008/0208070 A1
(43) Pub. Date: Aug. 28, 2008

(54) Defibrillator with Automatic Shock First/Cpr First Algorithm

(75) Inventors: David Snyder, Bainbridge Island, WA (US); Stacy Gehman, Seattle, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., Eindhoven (NL)

(21) Appl. No.: 11/917,272

(22) PCT Filed: Jun. 13, 2006
(86) PCT No.: PCT/IB06/51897

§ 371 Date: Dec. 12, 2007

Related U.S. Application Data
(60) Provisional application No. 60/693,746, filed on Jun. 23, 2005.
(60) Provisional application No. 60/751,269, filed on Dec. 16, 2005.

(51) Int. Cl. A61B 5/02; A61N 1/39

(52) U.S. Cl. 600/518; 607/5

(57) ABSTRACT

An automated external defibrillator (AED) with an improved rescue protocol is described which follows a “shock first” or a “CPR first” rescue protocol after identification of a treatable arrhythmia, depending upon an estimate of the probability of successful resuscitation made from an analysis of a patient parameter measured at the beginning of the rescue.

The invention relates generally to electrotherapy circuits, and more particularly, to a defibrillator which analyzes patient physiological data and determines whether a shock or CPR therapy should be conducted.

Defibrillators deliver a high-voltage impulse to the heart in order to restore normal rhythm and contractile function in patients who are experiencing arrhythmia, such as ventricular fibrillation (“VF”) or ventric ular tachycardia (“VT”) that is not accompanied by spontaneous circulation. There are several classes of defibrillators, including manual defibrillators, implantable defibrillators, and automatic external defibrillators (“AEDs”). AEDs differ from manual defibrillators in that AEDs can automatically analyze the electrocardiogram (“ECG”) rhythm to determine if defibrillation is necessary. In nearly all AED designs, the user is prompted to press a shock button to deliver the defibrillation shock to the patient when a shock is advised by the AED.

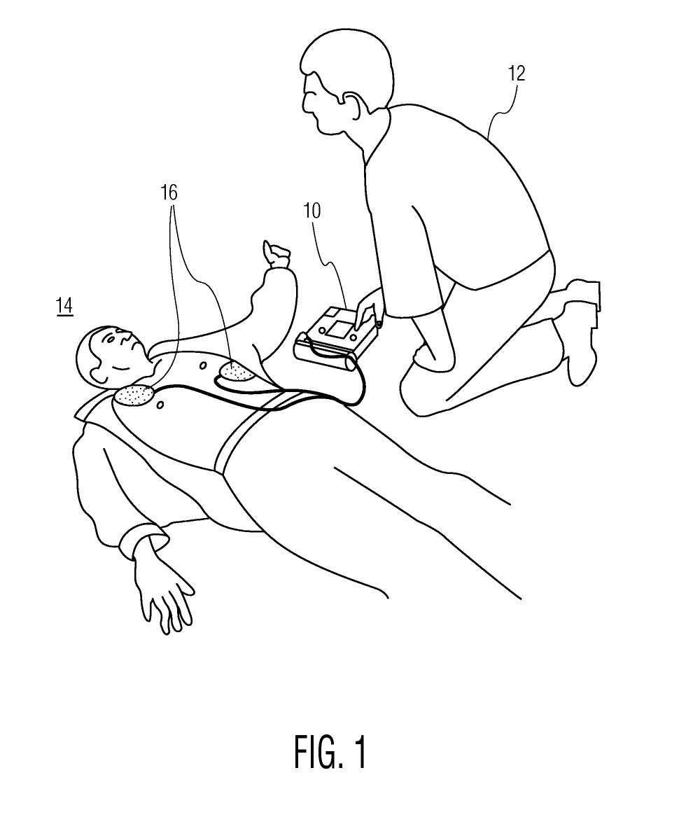

FIG. 1 is an illustration of a defibrillator 10 being applied by a user 12 to resuscitate a patient 14 suffering from cardiac arrest. In sudden cardiac arrest, the patient is stricken with a life threatening interruption to the normal heart rhythm, typically in the form of VF or VT that is not accompanied by spontaneous circulation (i.e., shockable VT). In VF, the normal rhythmic ventricular contractions are replaced by rapid, irregular twitching that results in ineffective and severely reduced pumping by the heart. If normal rhythm is not restored within a time frame commonly understood to be approximately 8 to 10 minutes, the patient will die. Conversely, the quicker that circulation can be restored (via CPR and defibrillation) after the onset of VF, the better the chances that the patient 14 will survive the event. The defibrillator 10 may be in the form of an AED capable of being used by a first responder. The defibrillator 10 may also be in the form of a manual defibrillator for use by paramedics or other highly trained medical personnel.

A pair of electrodes 16 are applied across the chest of the patient 14 by the user 12 in order to acquire an ECG signal from the patients heart. The defibrillator 10 t hen analyzes the ECG signal for signs of arrhythmia. If VF is detected, the defibrillator 10 signals the user 12 that a shock is advised. After detecting VF or other shockable rhythm, the user 12 then presses a shock button on the defibrillator 10 to deliver defibrillation pulse to resuscitate the patient 14.

Recent studies have shown that different patients may be resuscitated more effectively with different treatment regimens depending upon various factors. One factor which affects the likelihood of success of defibrillation is the amount of time that has elapsed since the patient experienced the arrhythmia. This research has indicated that, depending on the duration of cardiac arrest, a patient will have a better probability of recovery with one protocol as compared to another. If the AED is set up for a less effective protocol for the resuscitation of a particular patient, that patients probability of recovery may be reduced. These studies have shown that some of these patients have a better chance of being resuscitated if CPR is performed first, which will start by providing externally driven circulation which may bring the patient to a condition where application of a shock will be successful at restoring spontaneous circulation. Various attempts have been made to try to make this determination in an automated way from the patients vital signs. Since the determination of whether a shock is advised begins with analysis of the ECG waveform of the patient, these attempts have focused on analyzing the ECG waveform in order to make this determination. One line of studies has looked at the amplitude of the ECG waveform and found that patients with a stronger (higher amplitude) ECG waveform have a better chance of resuscitation with a defibrillating shock than do patients with a lower amplitude ECG. Since the amplitude of the ECG will generally decline with the passage of time after the onset of VF, this result is understandable. However, this measure is not a fail-proof predictor of resuscitation success. Another characteristic of the ECG which has been studied as a predictor of success is the frequency composition of the ECG waveform, with higher frequency content being found to correlate with resuscitation success. This analysis is done by performing a spectral analysis of the ECG waveform, as by using a fast Fourier transform processor to perform a spectral analysis of the ECG. This, too, has not been found to be a completely accurate predictor of success. Other researchers have multiplied amplitude and frequency information of the ECG with each other to produce a weighted high frequency measurement as a predictor of success, which takes advantage of both characteristics. Accordingly it is desirable to have a defibrillator determine a treatment regimen with a high probability of success automatically and with high accuracy. It is further desirable to determine the treatment regimen quickly, as soon as the AED is attached to the patient. Failure to do so can lead to several problems. If, for example, a rescuer arrives at the scene with an AED set up to perform CPR first (i.e. prior to defibrillation) and finds that good CPR is already in progress, a defibrillation shock is unnecessarily delayed. On the other hand, if a rescuer arrives at the scene with an AED set up to deliver a shock-first (i.e. prior to CPR) and finds a long-downtime patient with no CPR in progress, CPR may be delayed. In each of these situations, the less optimal rescue protocol may reduce the likelihood of survival.

In accordance with the principles of the present invention, a defibrillator is described which automatically analyzes an ECG waveform and produces a likelihood of return of spontaneous circulation (ROSC) score. The ROSC score is compared to a threshold to advise a treatment regimen which is more likely to be successful. The treatment regimen can be to shock the patient first, then analyze the ECG further and possibly provide CPR. Another possible treatment regimen is to provide CPR to the patient before delivering a shock. A defibrillator is described which implements the ROSC scoring processor in an efficient manner and which produces a ROSC score quickly and conveniently.

In the drawings:

FIG. 1 is an illustration of a defibrillator being applied to a patient suffering from cardiac arrest.

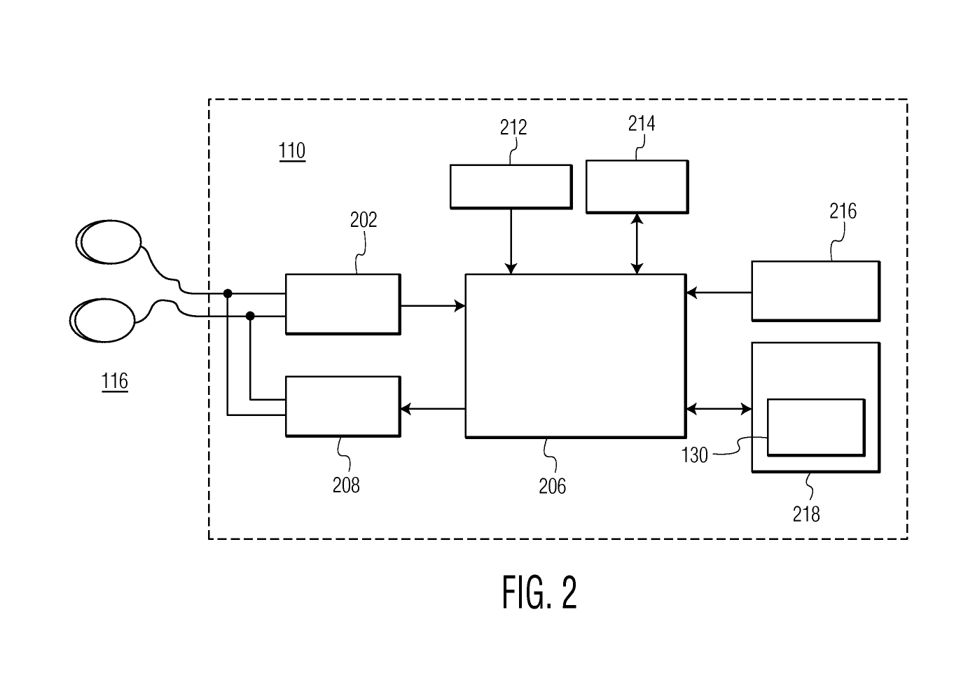

FIG. 2 is a block diagram of a defibrillator constructed in accordance with the principles of the present invention.

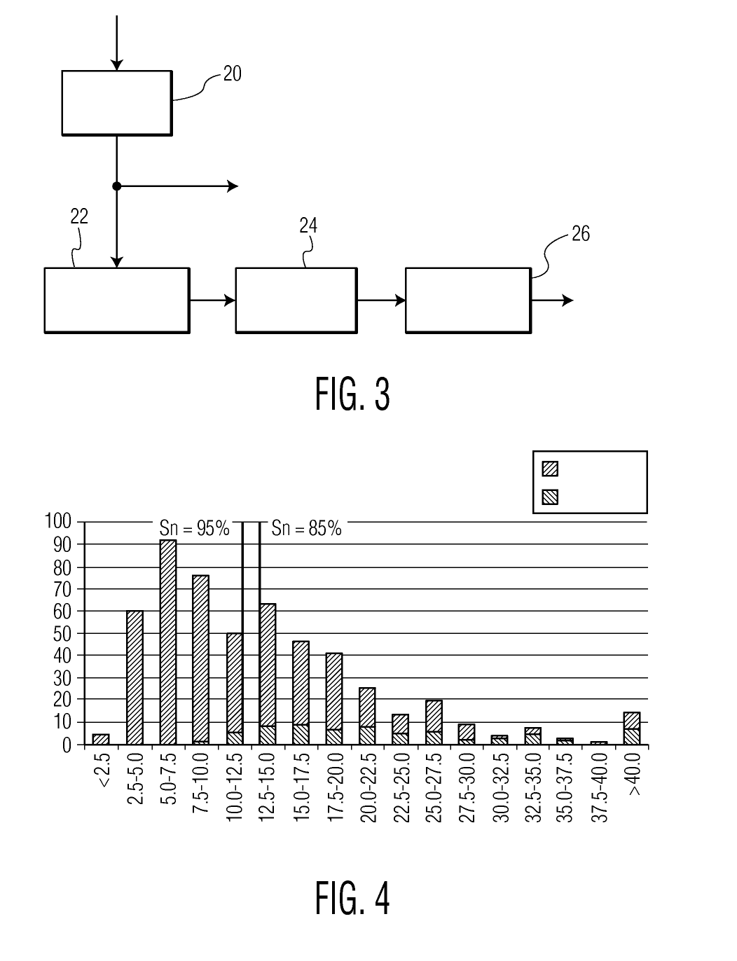

FIG. 3 is a is a detailed block diagram of a ROSC predictor constructed in accordance with the principles of the present invention.

FIG. 4 is a graph of patient data illustrating the determination of a threshold which can be used in the ROSC predictor of FIG. 3.

FIG. 2 illustrates a defibrillator 110 constructed in accordance with the principles of the present invention. For purposes of the discussion that follows, the defibrillator 110 is configured as an AED, and is designed for small physical size, light weight, and relatively simple user interface capable of being operated by personnel without high training levels or who otherwise would use the defibrillator 110 only infrequently. In contrast, a paramedic or clinical defibrillator of they type generally carried by an emergency medical service (EMS) responder tends to be larger, heavier, and have a more complex user interface capable of supporting a larger number of manual monitoring and analysis functions. Although the present embodiment of the invention is described with respect to application in an AED, other embodiments include application in different types of defibrillators, for example, manual defibrillators, and paramedic or clinical defibrillators.

An ECG front end circuit 202 is connected to a pair of electrodes 1 16 that are connected across the chest of the patient 14. The ECG front end circuit 202 operates to amplify, buffer, filter and digitize an electrical ECG signal generated by the patient's heart to produce a stream of digitized ECG samples. The digitized ECG samples are provided to a controller 206 that performs an analysis to detect VF, shockable VT or other shockable rhythm and, in accordance with the present invention, that performs an analysis to determine a treatment regimen which is likely to be successful. If a shockable rhythm is detected in combination with determination of a treatment regimen that indicates immediate defibrillation shock, the controller 206 sends a signal to HV (high voltage) delivery circuit 208 to charge in preparation for delivering a shock and a shock button on a user interface 214 is activated to begin flashing. When the user presses the shock button on the user interface 214 a defibrillation shock is delivered from the IV delivery circuit 208 to the patient 14 through the electrodes 116.

The controller 206 is coupled to further receive input from a microphone 212 to produce a voice strip. The analog audio signal from the microphone 212 is preferably digitized to produce a stream of digitized audio samples which may be stored as part of an event summary 130 in a memory 218. The user interface 214 may consist of a display, an audio speaker, and control buttons such as an on-off button and a shock button for providing user control as well as visual and audible prompts. A c lock 216 provides real-time clock data to the controller 206 for time-stamping information contained in the event summary 130. The memory 218, implemented either as on-board RAM, a removable memory card, or a combination of different memory technologies, operates to store the event summary 130 digitally as it is compiled over the treatment of the patient 14. The event summary 130 may include the streams of digitized ECG, audio samples, and other event data as previously described.

The AED of FIG. 2 has several treatment rescue protocols or treatment modes which may be selected during setup of the AED when it is initially received by the EMS service. One type of protocol is the “shock first” protocol. When the AED is set up for this protocol, the AED will, when connected to a patient and activated, immediately analyze the patient's ECG heart rhythm to make a heart rhythm classification. If the analysis determines that an arrhythmia treatable with electrical defibrillation is present, typically either ventricular fibrillation (VF) or pulseless ventricular tachycardia (VT), the rescuer is informed and enabled to deliver the shock. If it is determined that the arrhythmia is not treatable with a defibrillation shock, the AED will go into a “pause” mode during which CPR may be performed.

The second type of protocol is the “CPR first” protocol. When the AED is set up for this protocol, the AED will begin operating by instructing the rescuer to administer CPR to the patient. After CPR is administered for a prescribed period of time, the AED begins to analyze the ECG data to see if an arrhythmia treatable with electrical defibrillation is present.

In accordance with the principles of the present invention the AED 110 has a third setup, which is to initially recommend a treatment protocol, either shock first or CPR first. This is done by the AED which begins by analyzing the patient's ECG waveform, calculating and evaluating a ROSC score as described below. From the evaluation of the ROSC score a treatment protocol is recommended. The recommended protocol may be immediately carried out by the AED, or the recommendation presented to the rescuer for his or her final decision on the treatment protocol to be carried out.

FIG. 3 illustrates a portion of the ECG front end circuit 202 and controller 206 which operate in accordance with the principles of the present invention. As previously mentioned the electrodes 116 provide ECG signals from the patient which are sampled (digitized) by an A/D converter 20. The digitized ECG signals are coupled to the ECG analysis processor in the controller which analyzes the ECG waveform to determine whether application of a shock is advised. The ECG samples are coupled to a downsampler 22 which subsamples the stream of ECG samples to a lower data rate. For instance, a data stream of 200 samples/sec may be downsampled to 100 samples/sec. The downsampled ECG data is coupled to a ROSC calculator 24 which determines a ROSC score from the ECG data. The ROSC score is compared against a threshold by threshold comparator 26 to determine a mode of treatment which is most likely to lead to a successful resuscitation. This mode determination is coupled to the mode selection portion of the controller, which either selects the desired mode automatically or presents the mode as a recommendation to the rescuer who may then either decide to follow the recommended mode or an alternate treatment regimen.

The ROSC calculator 24 may be operated in several ways. For one example, the ROSC score is calculated as the mean magnitude of the bandwidth limited first derivative (or first difference, which is a discrete -time analog) of the ECG over a period of a few seconds. Since the bandwidth limited first derivative may already be calculated for arrhythmia detection by the controller 206, the additional computation may involve only the additional calculation of an average absolute value. This process can be implemented as a real -time measure by means of a moving average requiring only one addition and one subtraction per sample. For instance, the difference of successive samples may be taken for a stream of samples received over a period of 4.5 seconds at a 100 sample/sec rate. The signs of the differences are discarded to produce absolute values, which are summed over the 4.5 second period. This produces a ROSC score value which is equivalent to a frequency weighted average amplitude of the ECG waveform. The score may be scaled or further processed in accordance with the architecture and demands of the instant system.

Since the spectrum of the first derivative is proportional to frequency, the ROSC score is largely unaffected by CPR artifact, most of which will be very low frequency.

Another alternative way to calculate a mean value is to square t he differences of the consecutive samples, then sum the products and take the square root of the sum. This produces an RMS (root mean square) form of ROSC score.

As an alternative to the mean value computation, another approach is to use the median magnitude of the first derivative. This approach is more computationally intensive, but can advantageously be more robust to noise. Care must be taken to avoid de-emphasizing the signal that gives the measure its discriminating power. In another embodiment, a trimmed mean or min-max calculation can offer a favorable compromise. By eliminating the largest outliers, greater immunity to impulse artifacts (e.g. physical disturbances of the electrode pads) can be provided. By eliminating the largest outliers, the occasional high amplitude artifact which would occur relatively infrequently can be eliminated without significantly reducing the discriminating power associated with the data of cardiac origin.

An AED has been constructed to operate in accordance with the present invention. The implemented ROSC score processor has been found to identify ECG rhythms which result in ROSC following immediate defibrillation with high sensitivity, e.g., around 90%, and specificity greater than 60%. Sensitivity (Sn) is the percentage of patients that would achieve ROSC in response to an immediate defibrillation shock, that are correctly identified by the ROSC score. Specificity is the percentage of patients that would not achieve ROSC in response to an immediate defibrillation shock, that are correctly identified by the ROSC score. Sensitivity and specificity with respect to ROSC may be traded off in approximately equal proportion.

An implementation whereby alternative setup sensitivities were made available to the user is shown by the graph of FIG. 4. A database was assembled of the results of patients treated with defibrillation, some of whom achieved ROSC in response to an initial defibrillation shock and some of whom did not. The patients were treated after varying cardiac arrest durations. The ROSC score calculated by the implemented system was in the range of 2.5 to 40.0 units, where each unit corresponds to 0.25 mV/sec. The more lightly shaded portions of the bars in the graph indicate patients in the database who exhibited ROSC after delivery of a shock. The more darkly shaded portions of the bars indicate patients who did not exhibit ROSC after treatment. The graph shows the results of ROSC scoring by the system, which exhibited a 95% sensitivity to ROSC fol lowing an initial shock for patients with ROSC score greater than 3.0 mV/sec, and a sensitivity of 85% for patients with ROSC score greater than 3.6 mV/sec. Below a ROSC score of about 2.5 mV/sec, 100% of the patient population failed to achieve ROSC as the result of a first shock and may have benefited from a CPR first regimen of treatment. In the implemented system two thresholds of different sensitivities were used, one of 95% sensitivity and the other of 85% sensitivity. The user is thus able to sele ct a desired sensitivity during setup of the AED and can favor greater use of shock first with selection of the higher sensitivity (95%) or greater use of CPR first with a lower sensitivity (85%).

The implemented system has also been found to identify a go od outcome population for patients treated with a shocks-first protocol, experiencing neurologically intact survival of 53%, (95% CI [40%, 67%]). The implemented system also identified a poor outcome group that achieved neurologically intact survival of only 4%, (95% CI [0.1%, 20%]) and who might therefore benefit from CPR-first resuscitation.

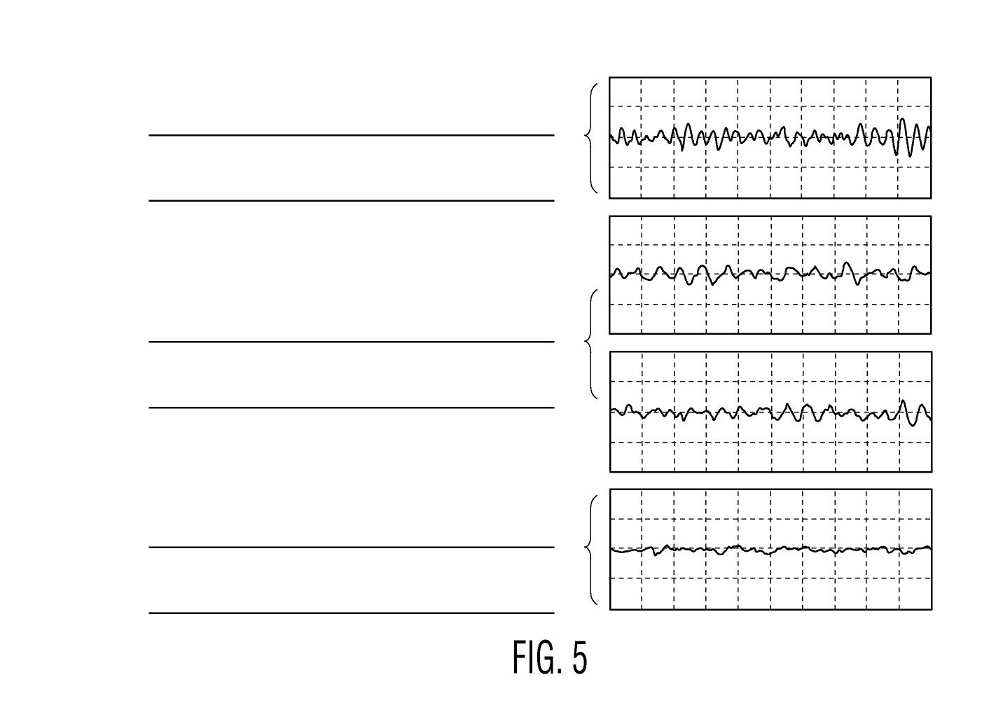

FIG. 5 illustrates the results obtained by the constructed system for four ECG waveforms with different sensitivity settings. In the Auto 1 (higher) sensitivity setting, a shock-first is advised in response to the first three ECG waveforms and CPR—first is advised for the fourth. In the Auto 2 (lower) sensitivity setting a shock-first is advised for the first ECG waveform and CPR -first is advised for the other three ECG waveforms.

1. a defibrillator comprising:
an ECG signal input coupled to a source of ECG signals;
an ECG data analysis circuit responsive to ECG signals which analyzes ECG data to determine whether a shock is recommended or a shock is not recommended;
a treatment decision processor responsive to ECG signal information which acts to estimate the probability of resuscitation from a shock; and
a defibrillator mode circuit responsive to the probability of resuscitation estimate which is operable to set the defibrillator to a shock mode or a CPR mode of operation.

2. The defibrillator of claim 1, wherein the ECG data analysis circuit determines that a shock is recommended when the presence of an arrhythmia is detected by the circuit.

3. The defibrillator of claim 1, wherein the treatment decision processor further comprises an analysis algorithm using a mean magnitude of the first derivative of ECG data.

4. The defibrillator of claim 1, wherein the treatment decision processor further comprises an analysis algorithm using a median magnitude of the first derivative of ECG data.

5. The defibrillator of claim 1, wherein the treatment decision processor further comprises an analysis algorithm producing an estimate of the probability of resuscitation which is a function of the frequency weighted average amplitude of ECG data.

6. The defibrillator of claim 5, wherein the analysis algorithm executes a ROSC scoring algorithm.

7. The defibrillator of claim 6, wherein the analysis algorithm further compares a ROSC score against a threshold.

8. The defibrillator of claim 7, wherein the threshold comprises a user adjustable sensitivity setting.

9. The defibrillator of claim 1, wherein the ECG data analysis circuit and the treatment decision processor are integrated into a common processor of the defibrillator.

10. The defibrillator of claim 9, wherein the treatment decision processor is responsive to ECG signal information produced by the ECG data analysis circuit.

11. The defibrillator of claim 1, wherein the defibrillator mode circuit is manually set by a user.

12. The defibrillator of claim 1, wherein the treatment decision processor is responsive to ECG signal information produced by the ECG data analysis circuit.

13. A method for delivering electrotherapy from a defibrillator comprising the steps of:
receiving patient ECG signals;
determining from the ECG signals the presence of an arrhythmia treatable by the defibrillator; and
estimating from ECG data, if the presence of a treatable arrhythmia is determined, the probability of resuscitation shock success; and
selecting the treatment protocol based upon the estimated probability of resuscitation.

14. The method of claim 13, wherein selecting further comprises selecting either a shock protocol or a CPR protocol.

15. The method of claim 13, wherein estimating further comprises executing an algorithm using a mean magnitude of the first derivative of the ECG data.

16. The method of claim 13, wherein estimating further comprises executing an algorithm using a median magnitude of the first derivative of the ECG data.

17. The method of claim 14, wherein determining further comprises determining whether a shock is advised.

18. The method of claim 13, wherein estimating further comprises computing a ROSC score.

19. The method of claim 18, wherein estimating further comprises comparing the ROSC score to a threshold.

20. The method of claim 19, further comprising selecting a threshold.

21. The method of claim 20, wherein selecting a threshold further comprises selecting a sensitivity-dependent threshold.

22. The method of claim 13, wherein the estimating and selecting steps are performed after the step of receiving patient ECG signals is initially performed.
